(19) United States
(12) Plant Patent Application Publication
(10) Pub. No.: US 2013/0031684 P1
(43) Pub. Date: Jan. 31, 2013

(54) Apple tree named 'Fujion'

(50) ;

(71) Applicants: Michelangelo Leis, Ferrara (IT); Alessio Martinelli, Ferrara (IT); Francesco Tagliani, Argenta (IT); Donata Azzolini, Quingentole (IT); Pietro Castagnoli, Quingentole (IT); Alessandro Castagnoli, Quingentole (IT)

(72) Inventors: Michelangelo Leis, Ferrara (IT); Alessio Martinelli, Ferrara (IT); Francesco Tagliani, Argenta (IT); Gianfranco Castagnoli, Quingentole (IT)

(73) Assignee: C.I.V. - CONSORZIO ITALIANO VIVAISTI - SOCIETA CONSORTILE A R.L.

(21) Appl. No.: 13/374,438

(22) Filed: Dec. 29, 2011

(30) Foreign Application Priority Data
Jul. 26, 2011 (QZ) 2011/1876

(51) Int. Cl. A01H 5/00

(52) U.S. Cl. PLT/168

(57) ABSTRACT

A new and distinct Malus domestica Mill. apple tree variety named ‘Fujion’ particularly characterized by having a medium vigorous tree, precocious fruit bearing with high productivity. Resistant to scab. Harvest period more concentrated than for Fuji and can generally be completed in two pickings.

The fruit is similar to ‘Fuji-Nagafu’ 12 with an intense red over colour and well-defined stripes. It is round and slightly elongated, very regular and more consistent in size than Fuji. The flesh is crispy, juicy and intensely sweet, generally sweeter than Fuji.

### LATIN NAME OF THE GENUS AND SPECIES OF THE PLANT CLAIMED

Malus domestica Mill.

### VARIETY DENOMINATION

‘Fujion’

### PRIORITY CLAIM

This application claims the benefit of European Community Plant Variety Office Plant Breeders' Rights Application No. 2011/1876, filed Jul. 26, 2011.

### BACKGROUND OF THE INVENTION

The present invention relates to a new and distinct variety of apple tree, botanically known as Malus domestica Mill. of the Family Rosaceae, and hereinafter referred to by the variety denomination ‘Fujion’.

The new Malus variety is a product of a planned breeding program conducted by the inventors, Michelangelo Leis, Alessio Martinelli, Gianfranco Castagnoli and Francesco Tagliani in S. Giuseppe di Comacchio (Ferrara), Italy. The objective of the breeding program was to develop new Malus varieties with improved production characteristics, high-quality flavour and aroma, and sustainability by increasing the tree's natural resistance. The primary objective of the Consorzio Italiano Vivaisti's research programme is to selection new apple varieties with natural resistances, in particular to scab.

The new Malus variety, ‘Fujion’, originated from a cross made in a planned, controlled breeding program in S. Giuseppe di Comacchio (Ferrara), Italy. The female parent is an unpatented C.I.V selection denominated ‘U7L-7’. The male parent is an unpatented C.I.V selection denominated ‘H-2’. ‘Fujion’ was discovered and selected in October 1993 by the inventors as a flowering plant within the progeny of the stated cross in a controlled environment in S. Giuseppe di Comacchio (Ferrara), Italy.

Asexual reproduction of the new Malus variety by budding and grafting was first performed in February 1994 and in the following years in S. Giuseppe di Comacchio (Ferrara), Italy, and has demonstrated that the combination of characteristics as herein disclosed for the new Malus variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘Fujion’ which in combination distinguish this apple tree as a new and distinct variety:

1. Bicolored variety intense red over colour with defined stripes;

2. Scab resistance;

3. Medium vigor, open habit with short branches, easy to manage;

4. High sugar content and

5. Long storage ability.

In comparison to the similar variety ‘Fuji-Nagafu 12’, ‘Fujion’ differs primarily in the traits listed in Table 1:

TABLE 1

| | Characteristic in | State of | State of |
|---|---|---|---|
| Denomination | which the similar | expression of | expression of |
| of similar variety | variety is different | similar variety | candidate variety |
| ‘Fuji - Nagafu 12’ | Relative area of | Small | Large |
| | over colour | | |
| | Tree vigour | Strong | Medium |
| | Resistance to | No | Scab resistant |
| | disease | | |

Of the commercial cultivars known to the inventors, the most similar in comparison to ‘Fujion’ is variety ‘Fuji-Nagafu 12’ as compared in Table 1.

### BRIEF DESCRIPTION OF THE DRAWINGS

The accompanying photographs illustrate the overall appearance of the new apple tree ‘Fujion’ showing the colors as true as is reasonably possible with color reproductions of this type. Color in the photographs may differ slightly from the color value cited in the detailed botanical description, which accurately describe the color of ‘Fujion’. In the photographs the labels show the breeder's reference ‘LH-59’.

FIG. 1: illustrates the plant of ‘Fujion’;

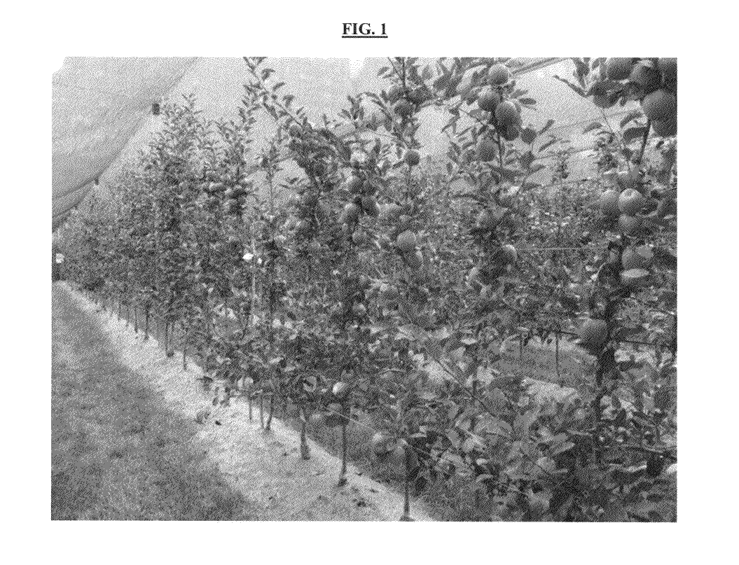

FIG. 2: illustrates the leaves of ‘Fujion’;

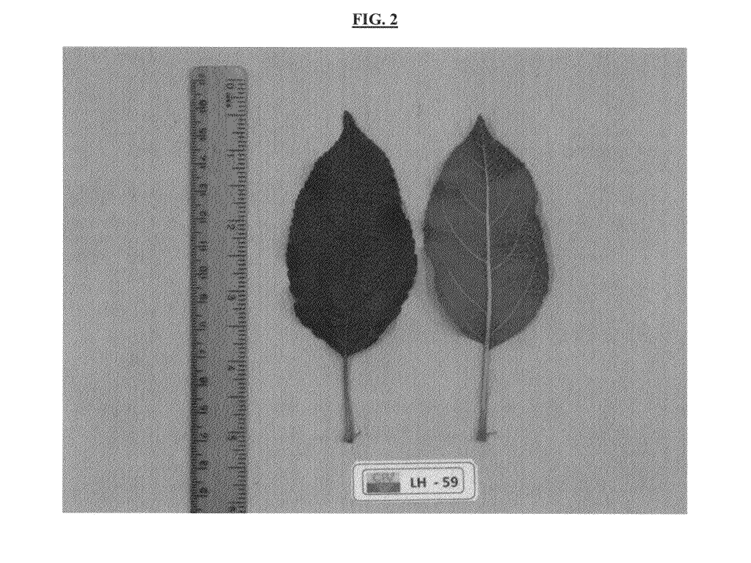

FIG. 3: illustrates the flowers of ‘Fujion’and

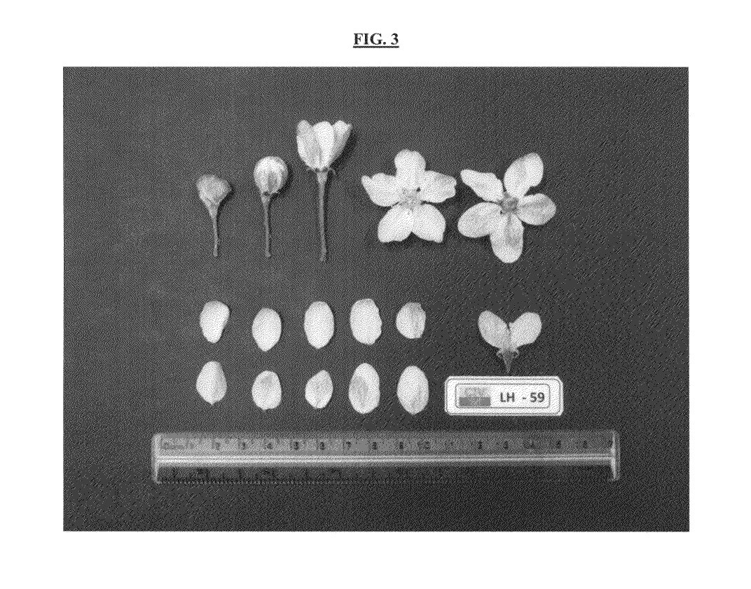

FIG. 4: illustrates the fruits of ‘Fujion’.

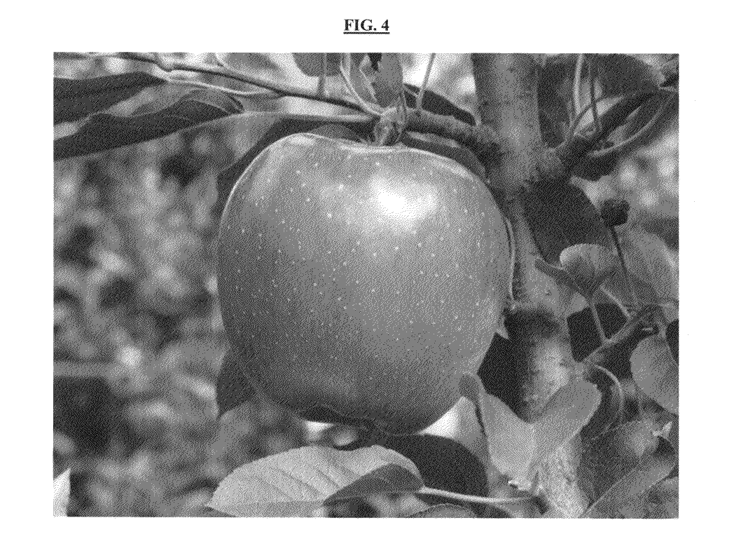

### DETAILED BOTANICAL DESCRIPTION

The new Malus variety ‘Fujion’ has not been observed under all possible environmental conditions. The phenotype of the new variety may vary with variations in environment such as temperature, light intensity, and day length without any change in the genotype of the plant.

The aforementioned photographs, together with the following observations, measurements and values describe the new Malus variety ‘Fujion’ as grown in the apple farm in S. Giuseppe di Comacchio (Ferrara), Italy, under conditions which closely approximate those generally used in commercial practice. The apple farm where ‘Fujion’ is grown is situated near the Adriatic sea (44° 45′ North, 12° 11′ East) and is zero (0) meters above sea level. The soil of the apple farm where ‘Fujion’ is grown is sandy, and the soil is treated with manure every year and irrigated with drip irrigation systems. The climate is temperate continental with high summer temperatures and low winter temperatures.

Unless otherwise stated, the detailed botanical description includes observations, measurements and values based on four (4) year old ‘Fujion’ trees that were grown in the apple farm in S. Giuseppe di Comacchio (Ferrara), Italy, from 2007 to 2010 All trees were of cropping maturity. Quantified measurements are expressed as an average of measurements taken from a number of individual trees of ‘Fujion’. The measurements of any individual tree, or any group of trees, of the new variety may vary from the stated average.

Color references are made to The Royal Horticultural Society Colour Chart (R.H.S.), except where general colors of ordinary significance are used. Color values were taken under daylight conditions at approximately at 10:00 am in S. Giuseppe di Comacchio (Ferrara), Italy.

All trees of ‘Fujion’, insofar as they have been observed, have been identical in all the characteristics described below.
Classification: Botanical: Malus domestica Mill.
Parentage: Female Parent: Malus domestica Mill. ‘U7L-7’ (unpatented C.I.V. selection) Male Parent: Malus domestica Mill. ‘H-2’ (unpatented C.I.V. selection)
Propagation: Budding and grafting on M9 rootstock.
Tree:
   Age.—Observed plants were four years old.
   Vigor.—medium vigor.
   Tree type.—ramified.
   Habit.—spreading, branches angle is typically 55 to 75° degrees from the vertical stem.
   Density.—medium density.
   Cropping behavior.—Precocious fruit bearing with high productivity.
   Type of bearing.—typically long shoot, presence of spur on 2-3 year old shoots.
   Production.—4^{th} year: about 16 Kg.
   Size.—Height: 2.7 m Spread: 1.30 m Trunk Diameter: 44 mm as measured 20 cm above point of grafting.
   Surface texture.—smooth.
   Bark color.—greyed-green RHS 197 B.
   Trunk lenticels.—.
   Overall shape.—round to elongated. Length: 1.8 mm Width: 1.2 mm Color: greyed-orange RHS 163 C. Density: 3 to 4 per cm².
   Branches.—Number per tree: about 25 at four (4) years. Length: Varies due to pyramidal shape of tree. At four (4) years, maximum of 100 cm to 120 cm; minimum of 40 cm to 60 cm. Diameter (at 3 years): About 10 mm to 14 mm. Surface texture: smooth. Color (at 3 years): grey, RHS 201 B. Internode length: About 2.5 cm to 3.5 cm. Internode diameter: About 9.0 mm to 12 mm.
   Branches lenticels.—Length: 0.9 mm Width: 0.5 mm Color: greyed-orange RHS 163 C. Density: About 5 per cm².
Leaves:
   Arrangement.—Alternate, simple, petiolated.
   Lamina.—Size: Length (4 year old): 84.6 mm (from 3^{rd} to 5^{th} fully expanded leaf). Width (4 year old): 44.7 mm (from 3^{rd} to 5^{th} fully expanded leaf). Length/width ratio: 1.89 Overall Shape: elliptical. Base shape: rounded. Apex shape: acuminate. Margin: serrate. Pubescence: absent on upper surface and weak pubescent on lower surface. Attitude in relation to shoot: outwards. Color (mature leaves): green, RHS 139 A on the upper side and 147 B underside. Color (immature leaves): green, RHS 141 A on the upper side and 138 A underside.
   Venation.—Type: prominent pinnate venation from a midrib to the edge. Color: yellow-green, RHS 148 C.
   Petiole.—Length: about 30 mm. Diameter: about 1.8 mm. Texture: hairy. Color: yellow-green, RHS 146 A with antocyanin coloration located on the base.
   Stipule.—Arrangement: adnate, grown together; medium size. Length (distance of stipules from basal attachment of petiole): average 3.75 mm Width: average 0.74 mm.
Spur: Present: on 2-3 year old shoots. Distance between each spur: On the two-three year old branches, the distance is about 20 mm to 40 mm
   Number of fruit per spur.—2 to 4 without thinning.
Flowers:
   Blooming time.—Full bloom on April 9^{th} at C.I.V. in S. Giuseppe di Comacchio (Ferrara) in year 2011.
   Blooming period.—9 to 11 days.
   Fragrance.—slight.
   Type.—inflorescence.
   Number of flowers per inflorescence.—5 to 6.
   Flower size.—Diameter: about 36 mm. Flower color: Primarily white, RHS 155B, when petals fully opened, undersides of petals there are shades red-purple color, RHS 70 C, making pink blooming stage pink, RHS 60 B.
   Buds.—Number of buds per spur: typically, one per spur. Shape: pointed Length: about 8 mm Width: about 4 mm. Color: brown, RHS 200 A with hairy surface.
   Petals.—Arrangement: intermediate. Number per flower: five (5). Length: average 15.58 mm. Width: average 11.52 mm. Length/width ratio: 1.35. Overall shape: elliptic. Apex shape: obtuse. Base shape: rounded. Texture: smooth. Margin: entire. Color (upper surface): white, RHS 155 D. Color (lower surface): white, RHS 155 D with shade red-purple RHS 70 D.
   Sepals.—Number per flower: five (5). Length: average 5.98 mm. Width: average 3.06 mm. Length/width ratio: 1.95. Overall shape: lanceolate. Apex shape: acute. Texture: hairy. Margin: entire. Color: green, RHS 138 C.
   Pedicel.—Length: 25 mm to 29 mm. Diameter: 2 to 3 mm. Texture: hairy. Color: green, RHS 138 C.
Fruit:
   Keeping quality.—the fruits can be stored in cold temperature conditions without loosing firmness and juiciness. It has a long shelf life, up to two weeks without loosing firmness and juiciness.
   Maturity when described.—ripe for eating.
   Maturity period after full bloom.—About 149 days in S. Giuseppe di Comacchio (Ferrara) Italy.
   Date of first and last pickings.—About 30 September and 10 October in S. Giuseppe di Comacchio (Ferrara) Italy (North 44° 45′ 46.2″, East 012° 11′ 31.9″) in year 2011.
   General shape.—obloid.
   Average weight.—284 g.
   Fruit size.—Average height: 71 mm. Average diameter (at widest point): 87.2 mm.
   Position of maximum diameter.—between ¾ of the height near stem end and half of the fruit.
   Height/diameter ratio.—0.81.
   Stem.—Length: about 28 mm. Diameter: 2.5 mm. Color: yellow-green, RHS 146 C.
   Stalk cavity.—Depth: average 16 mm. Width: average 40.6 mm.
   Eye basin.—Aperture of eye: fully open Depth: average 11 mm. Width: average 35.4 mm. Crowning at calyx end: moderate. Position of sepals: erect. Calyx tube: funnel form.
   Skin.—Thickness: medium. Texture: smooth. Bloom: moderate. Greasiness: absent or very weak. Firmness (at picking time): 7 to 8 kg/cm². Overcolor color: purple red. Percentage of skin surface with overcolor color: large Pattern of overcolor: weakly defined flush with strongly defined stripes. Intensity of overcolor: medium Ground color: yellow-green, RHS 145 A.
   Skin lenticels.—Length: about 0.7 mm. Width: about 0.4 mm. Color: greyed-orange, RHS 164 C. Density: about 3 per cm² in the central area of the surface of fruit. The number of lenticels increases in a direction toward the hollow of the calyx and the size of the lenticels decreases. Color: yellow-green group, RHS 150 D. Texture: firm, crisp and juicy. Aroma: intense. Eating quality: good with high level content in sugar. Sugar content (at picking time): 14 to 16° Brix. Acidity/Starch (at picking time): Acidity: 4 to 6 g/lt Malic acid/starch: 4 Laimburg scale 1 to 5.
   Core.—Symmetry of core: slightly asymmetric. Distinctness of core lines: evident. Locules: Number (per fruit): 5 (five). Length: 12 mm. Width: 4.5 mm. Form: closed or moderately open.
Seeds: Number per fruit: 8 to 10. Number per locule: about 2. Shape: narrow elliptic. Length: average 8.5 mm. Width: average 4.5 mm. Color: greyed-orange, RHS 164 A.
Reproductive organs:
   Androecium.—Stamens: Number per flower: about 20 (Twenty). Length: average 8.65 mm. Filament: Length: about 6.94 mm. Anther: Shape: ovoid, flat in the center. Length: 2.5 mm. Diameter: 1.71 mm. Color: yellow, RHS 10 B. Pollen: Amount: abundant. Color: yellow, RHS 13 C. Requirements: the crab apple “Malus Evereste” is good pollinator.
   Gynoecium.—Stigma: Shape: funnel shape with receptive surface on top. Length: 0.9 mm. Width: 0.5 mm. Color: yellow, RHS 145 B Style: Number per flower: 5 (Five). Length: about 10 mm. Width: about 0.3 mm. Color: yellow-green, RHS 145 C. Ovary: Length: about 3.5 mm. Width: about 2.5 mm. Color: yellow-green, RHS 144 A.
   Use.—fresh market.
   Sensitivity to disease/pests.—scab resistance.
   Winter hardiness.—tolerance to temperatures of −12° C. without observed damage to wood and buds of dormant apple trees; but open flowers and young fruitlets are killed by exposure to −3° C. to −5° C., depending on the length of exposure.
   Drought/heat tolerance.—good tolerance to heat, up to 40° C., growth is limited by drought periods without irrigation.
   Shipping/storage characteristics.—very good storability under ULO-conditions (1° C., 2% 0₂, 2% CO₂) for up to six (6) months.

We claim:

1. A new and distinct Malus domestica Mill. apple tree variety named ‘Fujion’, substantially as illustrated and described herein.
